(19) United States
(12) Patent Application Publication
(10) Pub. No.: US 2025/0258121 A1
(43) Pub. Date: Aug. 14, 2025

(54) GAS SENSOR

(71) Applicant: TDK Corporation, Tokyo (JP)

(72) Inventors: Makoto SHIBATA, Tokyo (JP); Takumi MATSUO, Tokyo (JP)

(21) Appl. No.: 19/009,227

(22) Filed: Jan. 3, 2025

(30) Foreign Application Priority Data
Feb. 14, 2024 (JP) 2024-020015

(51) Int. Cl. G01N 27/04; G01N 33/00

(52) U.S. Cl. CPC G01N 27/04; G01N 33/0027

(57) ABSTRACT

Disclosed herein is a gas sensor that includes: first and second temperature-sensitive elements connected in series; third and fourth temperature-sensitive elements connected in series; and signal processing circuit configured to detect a first detection voltage appearing at a first node in a first period during which the first and third temperature-sensitive elements are heated to a first temperature range and the second and fourth temperature-sensitive elements are heated to a second temperature range, detect a second detection voltage appearing at a second node in a second period during which the first and third temperature-sensitive elements are heated to the second temperature range and the second and fourth temperature-sensitive elements are heated to the first temperature range, and calculate a concentration of a gas to be detected based on the first detection voltage and the second detection voltage.

### CROSS-REFERENCE TO RELATED APPLICATION

This application claims the benefit of Japanese Patent Application No. 2024-020015, filed on Feb. 14, 2024, the entire disclosure of which is incorporated by reference herein.

### BACKGROUND OF THE ART

### Field of the Art

The present disclosure relates to a gas sensor and, more particularly, to a gas sensor provided with a temperature-sensitive element such as a thermistor.

### Description of Related Art

International Publication WO 2020/031517 discloses a gas sensor provided with two series-connected thermistors. The gas sensor described in this document heats first and second thermistors to first and second temperature ranges, respectively, during a first period and heats the first and second thermistors to the second and first temperature ranges, respectively, during a second period to make thermal histories of the two thermistors coincide with each other.

However, the gas sensor described in the above document cannot detect the concentration of a gas to be detected in the second period.

### SUMMARY

The present disclosure describes a technology for increasing a period during which a gas sensor provided with a temperature-sensitive element such as a thermistor can detect the concentration of a gas to be detected.

A gas sensor according to an aspect of the present disclosure includes: a first series circuit including a first temperature-sensitive element and a second temperature-sensitive element connected in series; a second series circuit including a third temperature-sensitive element and a fourth temperature-sensitive element connected in series; a first power supply circuit configured to apply voltage to the first series circuit; a second power supply circuit configured to apply voltage to the second series circuit; and a signal processing circuit. The signal processing circuit is configured to: detect a first detection voltage appearing at a first node at which the first temperature-sensitive element and the second temperature-sensitive element are connected in a first period during which the first temperature-sensitive element and a third temperature-sensitive element are heated to a first temperature range and the second temperature-sensitive element and the fourth temperature-sensitive element are heated to a second temperature range; detect a second detection voltage appearing at a second node at which the third temperature-sensitive element and the fourth temperature-sensitive element are connected in a second period during which the first temperature-sensitive element and the third temperature-sensitive element are heated to the second temperature range and the second temperature-sensitive and element the fourth temperature-sensitive element are heated to the first temperature range; and calculate a concentration of a gas to be detected based on the first detection voltage and the second detection voltage.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 1 according to a first embodiment of the technology according to the present disclosure;

FIG. 2 is a schematic plan view for explaining the device structure of the heater resistors MH1 and MH2 of the sensor parts S1 and S2;

FIG. 3 is a schematic plan view for explaining the device structure of the thermistors Rd1 to Rd4 of the sensor parts S1 and S2;

FIG. 4 is a schematic superimposed plan view of FIGS. 2 and 3 for explaining the device structure of the sensor parts S1 and S2;

FIG. 5 is a schematic plan view for explaining a modification of the device structure of the thermistors Rd1 to Rd4 of the sensor parts S1 and S2;

FIG. 6 is a flowchart for explaining the operation of the gas sensor 1;

FIG. 7 is a timing chart for explaining the operation of the gas sensor 1;

FIG. 8 is a flowchart for explaining the operation of the gas sensor 1 according to a modification;

FIG. 9A is a graph showing a change in the measurement result signal OUT obtained when there is an offset between the detection voltage Vgas1 appearing at a node N1 and the detection voltage Vgas1 appearing at a node N2;

FIG. 9B is a graph showing a change in the average value of the measurement result signal OUT obtained in a measurement at a certain point in time and the measurement result signal OUT obtained in the measurement immediately prior to that;

FIG. 10 is a circuit diagram illustrating the configuration of a gas sensor 2 according to a second embodiment of the technology according to the present disclosure;

FIG. 11 is a circuit diagram illustrating the configuration of a gas sensor 3 according to a third embodiment of the technology according to the present disclosure;

FIG. 12 is a circuit diagram illustrating the configuration of a gas sensor 4 according to a fourth embodiment of the technology according to the present disclosure;

FIG. 13 is a circuit diagram illustrating the configuration of a gas sensor 5 according to a fifth embodiment of the technology according to the present disclosure; and

FIG. 14 is a schematic plan view for explaining the device structure of the thermistors Rd1 to Rd4 of the sensor parts S1 and S2 in the gas sensor 5.

### DETAILED DESCRIPTION OF THE EMBODIMENTS

Some embodiments of the present disclosure will be explained below in detail with reference to the accompanying drawings.

### First Embodiment

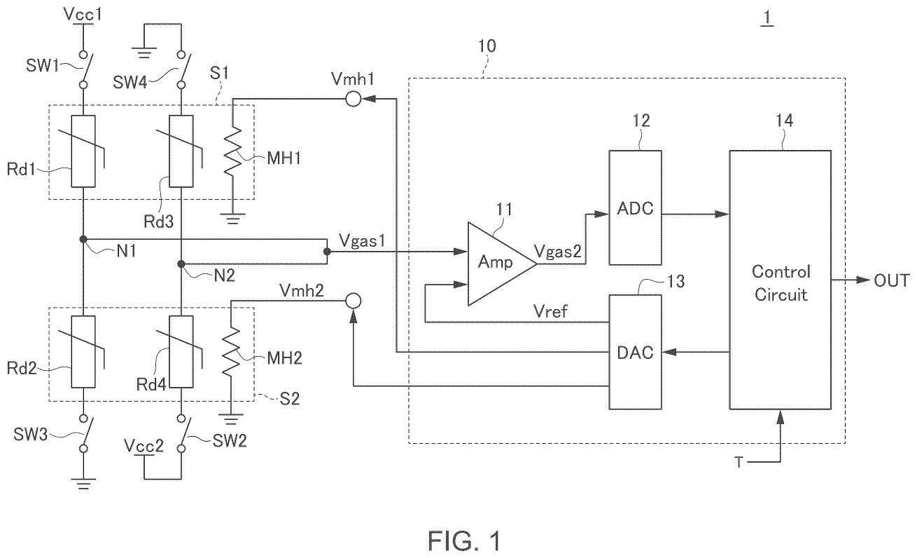

FIG. 1 is a circuit diagram illustrating the configuration of a gas sensor 1 according to a first embodiment of the technology according to the present disclosure.

As illustrated in FIG. 1, the gas sensor 1 according to the first embodiment has sensor parts S1 and S2 and a signal processing circuit 10. Although not particularly limited, the gas sensor 1 according to the present embodiment is a heat-conduction type gas sensor for detecting the concentration of CO₂ gas in the atmosphere.

The sensor part S1 includes thermistors Rd1, Rd3 and a heater resistor MH1. The sensor part S2 includes thermistors Rd2, Rd4 and a heater resistor MH2. The thermistors Rd1 to Rd4 all have a negative temperature coefficient of resistance. The thermistors Rd1 and Rd2 are connected in series to constitute a first series circuit. The thermistors Rd3 and Rd4 are connected in series to constitute a second series circuit. One end of the thermistor Rd1 and one end of the thermistor Rd2 are short-circuited by a node N1. One end of the thermistor Rd3 and one end of the thermistor Rd4 are short-circuited by a node N2. In the example illustrated in FIG. 1, the nodes N1 and N2 are short-circuited.

The other end of the thermistor Rd1 is connected, through a switch SW1, to a line to which a power supply voltage Vcc1 is supplied. The other end of the thermistor Rd2 is connected to a ground line through a switch SW3. The other end of the thermistor Rd3 is connected to a ground line through a switch SW4. The other end of the thermistor Rd4 is connected, through a switch SW2, to a line to which a power supply voltage Vcc2 is supplied. The power supply voltages Vcc1 and Vcc2 may have the same level. The switches SW1, SW3, the line to which the power supply voltage Vcc1 is supplied, and the ground line constitute a first power supply circuit that applies voltage to the first series circuit constituted by the thermistors Rd1 and Rd2. The switches SW2, SW4, the line to which the power supply voltage Vcc2 is supplied, and the ground line constitute a second power supply circuit that applies voltage to the second series circuit constituted by the thermistors Rd3 and Rd4. In the first series circuit, the connection relation of the thermistors Rd1 and Rd2 to the power supply voltage Vcc1 and ground line may be reversed. That is, the other end of the thermistor Rd1 may be connected to the ground line through the switch SW1, and the other end of the thermistor Rd2 may be connected, through the switch SW3, to the line to which the power supply voltage Vcc1 is supplied. Similarly, in the second series circuit, the connection relation of the thermistors Rd3 and Rd4 to the power supply voltage Vcc2 and ground line may be reversed. That is, the other end of the thermistor Rd3 may be connected, through the switch SW4, to the line to which the power supply voltage Vcc2 is supplied, and the other end of the thermistor Rd4 may be connected to the ground line through the switch SW2.

The resistance values of the thermistors Rd1 and Rd4 each serving as a temperature-sensitive element for detection are designed so as to fall within a first resistance range when the thermistors Rd1 and Rd4 are heated to a first temperature range (e.g., about 150° C.). The first temperature range is, for example, a predetermined temperature range included in the range of 100° C. or more and 230° C. or less, for example, a temperature range about 150° C. The resistance values of the thermistors Rd2 and Rd3 each serving as a temperature-sensitive element for reference are designed so as to fall within a second resistance range when the thermistors Rd2 and Rd3 are heated to a second temperature range (e.g., about 300° C.). The second temperature range is, for example, a predetermined temperature range included in the range of 250° C. or more and 450° C. or less, for example, a temperature range about 300° C. In the specification, the “temperature range” has a temperature width of, for example, within 1° C. For example, a temperature range about 150° C. may be a range of 149.5° C. or more and 150.5° C. or less. Also, for example, a temperature range about 300° C. may be a range of 299.5° C. or more and 300.5° C. or less. In the present embodiment, the second temperature range is set higher than the first temperature range. The first and second resistance ranges may partially overlap each other or may completely coincide with each other. When the first and second resistance ranges partially overlap each other or completely coincide with each other, it is possible to bring the level of a detection voltage Vgas1 appearing at the nodes N1 and N2 close to Vcc1/2 or Vcc2/2, achieving a wide dynamic range. The detection voltage Vgas1 appearing at the nodes N1 and N2 is supplied to the signal processing circuit 10.

When CO₂ gas is present in the measuring atmosphere in a state where the thermistors Rd1 and Rd4 as the temperature-sensitive element for detection is heated to around 150° C., heat dissipation characteristics of the thermistors Rd1 and Rd4 change according to the concentration of the CO₂ gas. This change appears as a change in the temperatures of the thermistors Rd1 and Rd4, i.e., the resistance values thereof. Specifically, CO₂ gas is lower in heat dissipation than air, so that the temperatures of the thermistors Rd1 and Rd4 increase as the concentration of CO₂ gas becomes high. Thus, for example, assuming that heating is performed so that temperatures of the thermistors Rd1 and Rd4 become 150° C. when CO₂ gas concentration in the measuring atmosphere is the same as CO₂ average concentration in the atmosphere, the temperatures of the thermistors Rd1 and Rd4 exceed 150° C. when CO₂ gas concentration in the measuring atmosphere is higher than CO₂ average concentration in the atmosphere. As a result, the resistance values of the thermistors Rd1 and Rd4 lower as compared with when CO₂ gas concentration in the measuring atmosphere assumes CO₂ average concentration in the atmosphere.

On the other hand, when CO₂ gas is present in the measuring atmosphere in a state where the thermistors Rd2 and Rd3 as the thermosensitive element for reference is heated to around 300° C., heat dissipation characteristics of the thermistor Rd2 and RD3 hardly change according to the concentration thereof, and the temperatures of the thermistors Rd2 and Rd3 also hardly change. Accordingly, a change in the resistance values of the thermistors Rd1 and Rd3 heated to around 300° C. according to the CO₂ gas concentration is sufficiently smaller than a change in the resistance values of the thermistors Rd1 and Rd4 heated to around 150° C. according to the CO₂ gas concentration and may be imperceptible. As a result, when the thermistors Rd1 and Rd2 are heated to around 150° C. and 300° C., respectively, the detection voltage Vgas1 corresponding to the concentration of CO₂ gas in the measuring atmosphere appears at the node N1. Similarly, when the thermistors Rd4 and Rd3 are heated to around 150° C. and 300° C., respectively, the detection voltage Vgas1 corresponding to the concentration of CO₂ gas in the measuring atmosphere appears at the node N2. Even when another gas, in which there is no significant difference between heat dissipation characteristics exhibited when the thermistors Rd1 to Rd4 are heated to around 150° C. and heat dissipation characteristics exhibited when the thermistors Rd1 to Rd4 are heated to around 300° C., is contained in the measuring atmosphere, the concentration of this gas has little influence on the detection voltage Vgas1. Thus, it is possible to selectively detect the concentration of CO₂ gas.

The signal processing circuit 10 has an amplifier 11, an AD converter (ADC) 12, a DA converter (DAC) 13, and a control circuit 14. The amplifier 11 compares the detection voltage Vgas1 and a reference voltage Vref to generate an amplified detection voltage Vgas2. The amplifier 11 is a differential amplifier, for example, and amplifies a difference between the detection voltage Vgas1 and the reference voltage Vref to generate and output the detection voltage Vgas2. The detection voltage Vgas2 is input to the AD converter 12. The AD converter 12 AD-converts the detection voltage Vgas2 into a digital detection voltage Vgas2 and supplies the digital detection voltage Vgas2 to the control circuit 14.

The control circuit 14 supplies various control parameters to the DA converter 13 in the form of digital values. The DA converter 13 D-A coverts the digital control parameters to generate heater voltages Vmh1 and Vmh2 and a reference voltage Vref. The heater voltage Vmh1 is applied to the heater resistor MH1 to heat the thermistors Rd1 and Rd3. The heater voltage Vmh2 is applied to the heater resistor MH2 to heat the thermistors Rd2 and Rd4. The reference voltage Vref is supplied to the amplifier 11.

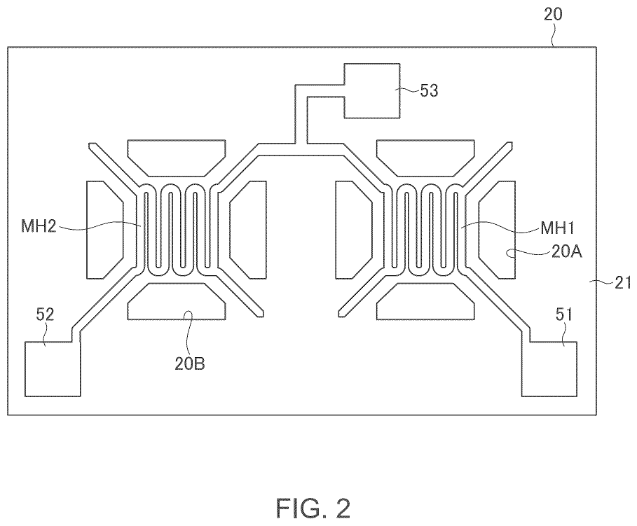

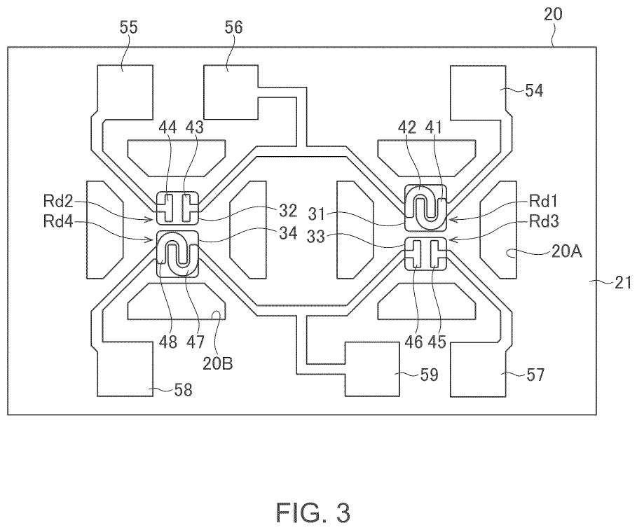

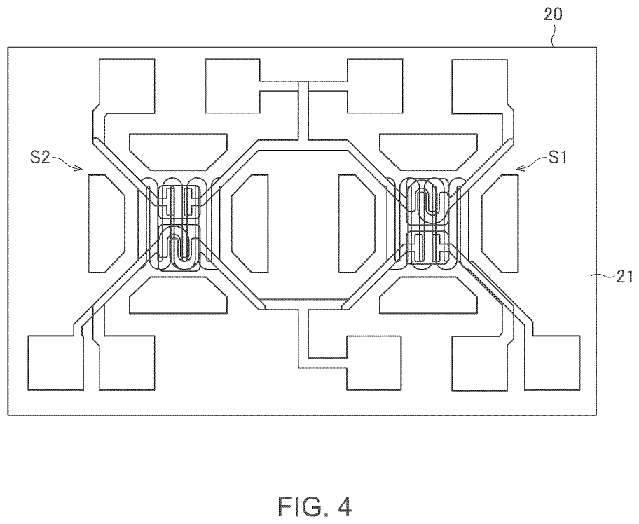

FIGS. 2 to 4 are schematic plan views for explaining the device structure of the sensor parts S1 and S2. FIG. 2 illustrates a portion corresponding to the heater resistors MH1 and and MH2, FIG. 3 illustrates a portion corresponding to the thermistors Rd1 to Rd4, and FIG. 4 is a superimposed view of FIGS. 2 and 3.

As illustrated in FIGS. 2 to 4, the sensor parts S1 and S2 include a substrate 20, an insulating film 21 formed on the surface of the substrate 20, the heater resistors MH1 and MH2 provided on the insulating film 21, thermistor films 31 and 33 formed so as to overlap the heater resistor MH1, thermistor films 32 and 34 formed so as to overlap the heater resistor MH2, a pair of counter electrodes 41 and 42 contacting the thermistor film 31, a pair of counter electrodes 43 and 44 contacting the thermistor film 32, a pair of counter electrodes 45 and 46 contacting the thermistor film 33, and a pair of counter electrodes 47 and 48 contacting the thermistor film 34. A first portion of the thermistor film 31 that is positioned between the pair of counter electrodes 41 and 42 and the counter electrodes 41 and 42 constitute the thermistor Rd1. A first portion of the thermistor film 32 that is positioned between the pair of counter electrodes 43 and 44 and the counter electrodes 43 and 44 constitute the thermistor Rd2. A first portion of the thermistor r film 33 that is positioned between the pair of counter electrodes 45 and 46 and the counter electrodes 45 and 46 constitute the thermistor Rd3. A first portion of the thermistor film 34 that is positioned between the pair of counter electrodes 47 and 48 and the counter electrodes 47 and 48 constitute the thermistor Rd4.

The substrate 20 is not particularly limited in material as long as it has adequate mechanical strength and is suitable for fine processing such as etching. Examples of the material of the substrate 20 include a silicon single crystal substrate, a sapphire single crystal substrate, a ceramic substrate, a quartz substrate, and a glass substrate. The substrate 20 and insulating film 21 are removed at notches 20A and 20B, and cavities are formed Further, the substrate 20 is removed in an area there. surrounded by the notches 20A and that surrounded by the notches 20B, and the heater resistors KH1 and MH2 and thermistors Rd1 to Rd4 are supported by the thin insulating film 21 in these areas. Such a membrane structure can suppress conduction of heat from the heater resistors MH1 and MH2 to the substrate 20.

The heater resistors MH1 and MH2 are each made of a conductive substance and may be made of a metal material having a comparatively high melting point, such as molybdenum (Mo), platinum (Pt), gold (Au), tungsten (W), tantalum (Ta), palladium (Pd), iridium (Ir), nickel (Ni), chrome (Cr), or an alloy containing two or more of them. As illustrated in FIG. 2, one end of the heater resistor MH1 is connected to a terminal electrode 51, and the other end thereof is connected to a terminal electrode 53. One end of the heater resistor MH2 is connected to a terminal electrode 52, and the other end thereof is connected to the terminal electrode 53. The heater resistors MH1 and MH2 may each have a meander shape.

The thermistor films 31 to 34 are each made of a material having a negative temperature coefficient of resistance, such as a composite metal oxide, amorphous silicon, polysilicon, or germanium. The counter electrodes 41 and 42 contacting the thermistor film 31 are connected to terminal electrodes 54 and 56, respectively. As a result, the terminal electrodes 54 and 56 are connected to each other through the thermistor film 31 positioned between the counter electrodes 41 and 42. The counter electrodes 43 and 44 contacting the thermistor film 32 are connected to terminal electrodes 56 and 55, respectively. As a result, the terminal electrodes 55 and 56 are connected to each other through the thermistor film 32 positioned between the counter electrodes 43 and 44. The counter electrodes 45 and 46 contacting the thermistor film 33 are connected to terminal electrodes 57 and 59, respectively. As a result, the terminal electrodes 57 and 59 are connected to each other through the thermistor film 33 positioned between the counter electrodes 45 and 46. The counter electrodes 47 and 48 contacting the thermistor film 34 are connected to terminal electrodes 59 and 58, respectively. As a result, the terminal electrodes 58 and 59 are connected to each other through the thermistor film 34 positioned between the counter electrodes 47 and 48.

The terminal electrode 54 is connected, through the switch SW1, to the line to which the power supply voltage Vcc1 is supplied. The terminal electrode 55 is connected to the ground line through the switch SW3. The terminal electrode 58 is connected, through the switch SW2, to the line to which the power supply voltage Vcc2 is supplied. The terminal electrode 57 is connected to the ground line through the switch SW4. The switches SW1 to SW4 may constitute a part of the signal processing circuit 10. The terminal electrode 56 constitutes the node N1. The terminal electrode 59 constitutes the node N2.

As illustrated in FIG. 3, the counter electrodes 41 and 42 (also counter electrodes 47 and 48) are so shaped as to be engaged with each other in a comb-like shape, while the counter electrodes 43 and 44 (also counter electrodes 45 and 46) are so shaped as to linearly face each other. As a result, the opposing length between the counter electrodes 41 and 42 (also counter electrodes 47 and 48) is longer than the opposing length between the counter electrodes 43 and 44 (also counter electrodes and 45 and 46). Further, the inter-electrode distance (opposing width) between the counter electrodes 41 and 42 (also counter electrodes 47 and 48) is smaller than the inter-electrode distance (opposing width) between the counter electrodes 43 and 44 (also counter electrodes 45 and 46). Thus, when the thermistor films 31 to 34 having substantially the same resistivity are heated to substantially the same temperature, the resistance value of the thermistor film 31 positioned between the counter electrodes 41 and 42 and that of the thermistor film 34 positioned between the counter electrodes 47 and 48 become lower than the resistance value of the thermistor film 32 positioned between the counter electrodes 43 and 44 and that of the thermistor film 33 positioned between the counter electrodes 45 and 46. The reason for this is as follows. As described above, the resistance values of the thermistors Rd1 and Rd4 as the temperature-sensitive element for detection are designed to fall within the first resistance range when the thermistors Rd1 and Rd4 are heated to about 150° C., while the resistance values of the thermistors Rd2 and Rd4 as the temperature-sensitive element for reference are designed to fall within the second resistance range when the thermistors Rd2 and Rd3 are heated to about 300° C., so that it is necessary to set the resistance values of the thermistors Rd1 and Rd4 lower than the resistance values of the thermistors Rd2 and Rd3 under the condition of the same temperature in order to make the first and second resistance ranges overlap or coincide with each other.

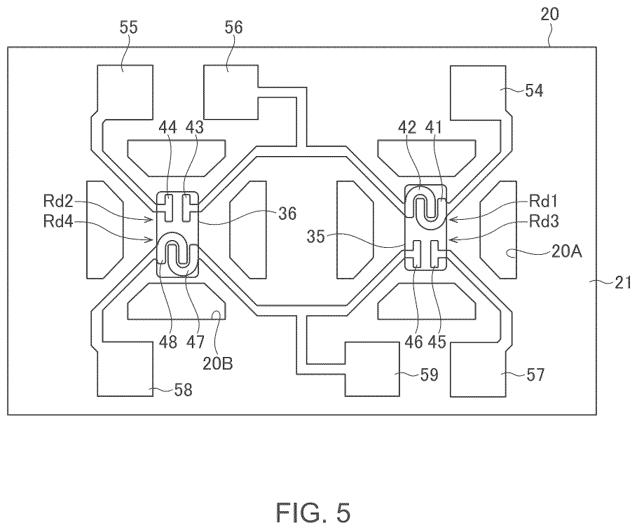

In the example illustrated in FIG. 3, the thermistor films 31 and 33 are separated from each other, and the thermistor films 32 and 34 are also separated from each other; however, as in the example illustrated in FIG. 5, it is possible to use a common thermistor film 35 for the counter electrodes 41 and 42 and counter electrodes 45 and 46 and a common thermistor film 36 for the counter electrodes 43 and 44 and counter electrodes 47 and 48. In this case, a first portion of the thermistor film 35 that is positioned between the pair of counter electrodes 41 and 42 and the counter electrodes 41 and 42 constitute the thermistor Rd1, a first portion of the thermistor film 36 that is positioned between the pair of counter electrodes 43 and 44 and the counter electrodes 43 and 44 constitute the thermistor Rd2, a second portion of the thermistor film 35 that is positioned between the pair of counter electrodes 45 and 46 and the counter electrodes 45 and 46 constitute the thermistor Rd3, and a second portion of the thermistor film 36 that is positioned between the pair of counter electrodes 47 and 48 and the counter electrodes 47 and 48 constitute the thermistor Rd4.

The following describes the operation of the gas sensor 1 according to the first embodiment.

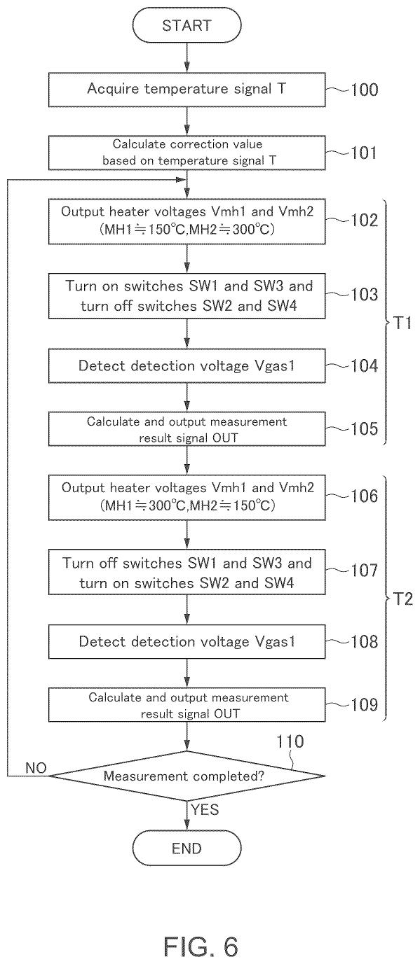

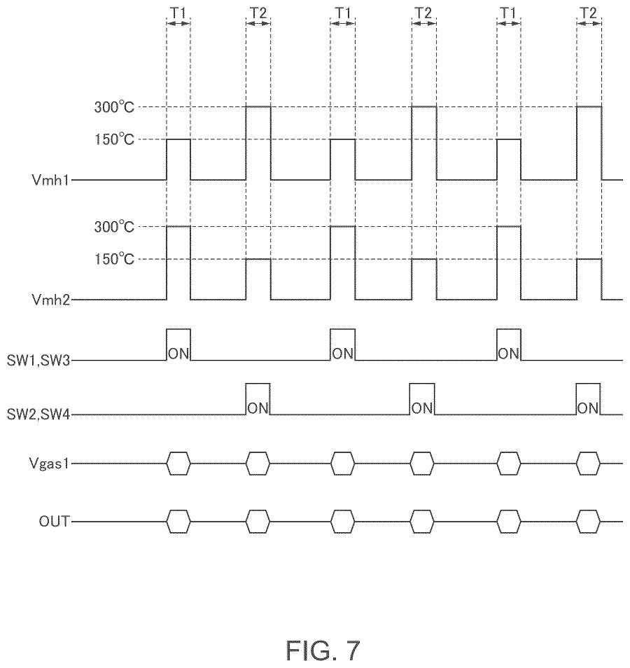

FIG. 6 is a flowchart for explaining the operation of the gas sensor 1. FIG. 7 is a timing chart for explaining the operation of the gas sensor 1.

The signal processing circuit 10 included in the gas sensor 1 acquires a temperature signal T indicating a temperature in the measuring atmosphere (step 100) and calculates a correction value based on the temperature signal T (step 101). The calculated correction value is used for cancelling offsets of the heater voltages Vmh1 and Vmh2 and reference voltage Vref due to the temperature in the measuring atmosphere.

Then, the signal processing circuit 10 performs operation steps in a period T1. In the period T1, the signal processing circuit 10 outputs the heater voltages that have been corrected based on the Vmh1 and Vmh2 correction value (step 102). In the period T1, the heater resistors MH1 and MH2 are heated so that the thermistors Rd1 and Rd3 become about 150° C. and that the thermistors Rd2 and Rd4 become about 300° C. For example, the heater voltages Vmh1 and Vmh2 are set so as to heat the thermistors Rd1 and Rd3 to 150° C. and heat the thermistors Rd2 and Rd4 to 300° C. when the concentration of CO₂ gas in the measuring atmosphere is the same as CO₂ average concentration in the atmosphere. In this state, the signal processing circuit 10 turns on the switches SW1 and SW3 and turns off the switches SW2 and SW4 (step 103). As a result, the detection voltage Vgas1 corresponding to the CO₂ gas concentration in the measuring atmosphere appears at the node N1. The detection voltage Vgas1 is fetched in the signal processing circuit 10 (step 104), and then the control circuit 14 included in the signal processing circuit 10 calculates a measurement result signal OUT indicating CO₂ gas concentration and externally outputs it (step 105). The thermistors Rd3 and Rd4 are heated in the period T1; however, the switches SW2 and SW4 are in an off state in the period T1, so that the thermistors Rd3 and Rd4 have no influence on the detection voltage Vgas1.

Then, the signal processing circuit 10 performs operation steps in a period T2. In the period T2, the signal processing circuit 10 outputs the heater voltages Vmh1 and Vmh2 that have been corrected based on the correction value (step 106). In the period T2, the heater resistors MH1 and MH2 are heated so that the thermistors Rd1 and Rd3 become about 300° C. and that the thermistors Rd2 and Rd4 become about 150° C. For example, the heater voltages Vmh1 and Vmh2 are set so as to heat the thermistors Rd1 and Rd3 to 300° C. and heat the thermistors Rd2 and Rd4 to 150° C. when the concentration of CO₂ gas in the measuring atmosphere is the same as CO₂ average concentration in the atmosphere. In this state, the signal processing circuit 10 turns off the switches SW1 and SW3 and turns on the switches SW2 and SW4 (step 107). As a result, the detection voltage Vgas1 corresponding to the CO₂ gas concentration in the measuring atmosphere appears at the node N2. The detection voltage Vgas1 is fetched in the signal processing circuit 10 (step 108), and then the control circuit 14 included in the signal processing circuit 10 calculates the measurement result signal OUT indicating CO₂ gas concentration and externally outputs it (step 109). The thermistors Rd1 and Rd2 are heated in the period T2; however, the switches SW1 and SW3 are in an off state in the period T2, so that the thermistors Rd1 and Rd2 have no influence on the detection voltage Vgas1.

By repeating the above-described steps 102 to 109, it is possible to periodically acquire the measurement result signal OUT (step 110).

As described above, in the operation of the gas sensor 1 according to the present embodiment, the period T1 during which the thermistors Rd1 and Rd3 are heated to about 150° C. and the thermistors Rd2 and Rd4 are heated to about 300° C. and the period T2 during which the thermistors Rd1 and Rd3 are heated to about 300° C. and the thermistors Rd2 and Rd4 are heated to about 150° C. are alternately repeated. This makes it possible to acquire the measurement result signal OUT in both the periods T1 and T2 while making the thermal histories of the thermistors Rd1 and Rd2 coincide with each other and thermal histories of the thermistors Rd3 and Rd4 coincide with each other. The periods T1 and T2 need not necessarily be alternately repeated; alternatively, a set of operation steps in the period T1 is performed plural times, and successively a set of operations in the period T2 is performed plural times.

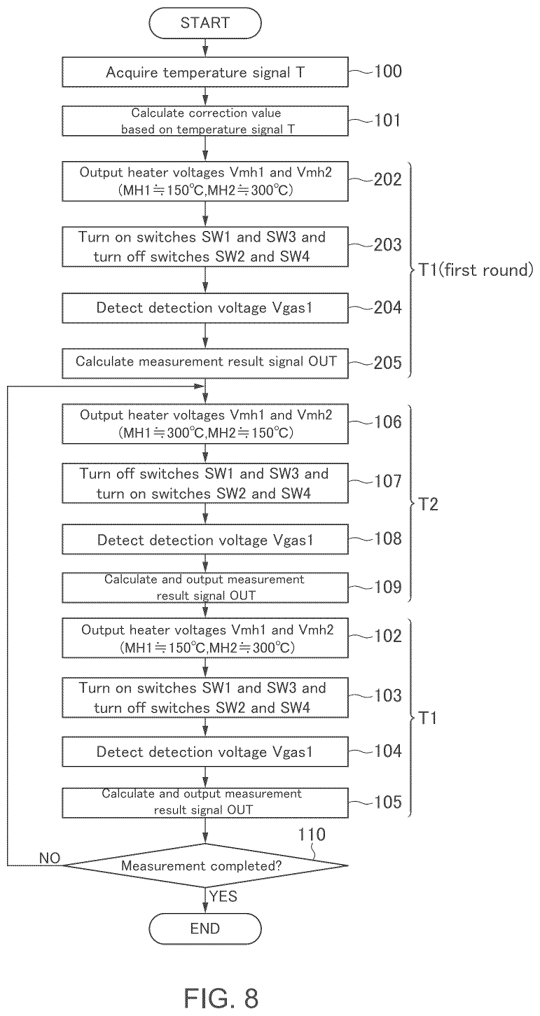

FIG. 8 is a flowchart for explaining the operation of the gas sensor 1 according to a modification. The same steps as those in the flowchart of FIG. 6 are denoted by the same step numbers.

After acquiring the temperature signal T (step 100) and calculating the correction value (step 101), the signal processing circuit 10 performs a first round of the period T1. The operation steps (steps 202 to 205) in the first round of the period T1 are basically the same as steps 102 to 105 described above; however, in step 205, the measurement result signal OUT is only calculated but is not externally output.

Then, the signal processing circuit 10 performs operation steps in the period T2. The operation steps (steps 106 to 109) in the period T2 according to the modification are basically the same as above-described steps 106 to 109; however, in step S109 according to the modification, the average value of the measurement result signal OUT calculated in step 205 and measurement result signal OUT calculated in step 109 is externally output.

Then, the signal processing circuit 10 performs operation steps in the period T1. The operation steps in the period T1 are basically the same as above-described steps 102 to 105; however, in step 105 according to the modification, the average value of the measurement result signal OUT calculated in step 109 and the measurement result signal OUT calculated in step 105 is externally output.

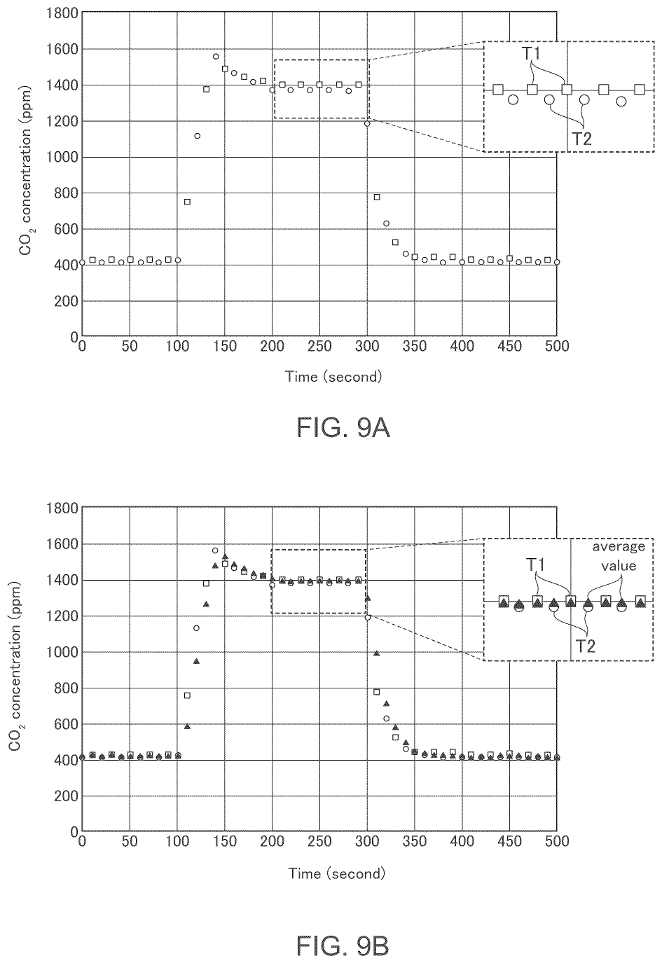

As described above, in the example of FIG. 8, the average value of the measurement result signal OUT acquired in the measurement performed at a certain point of time and the measurement result signal acquired in the immediately preceding measurement is externally output, so that, as illustrated in FIG. 9A for example, even when there exists a certain offset between the detection voltage Vgas1 appearing at the node N1 and the detection voltage Vgas1 appearing at the node N2, the offset does not appear in the measurement result signal OUT to be actually output, as illustrated in FIG. 9B. Although the moving average of two measurement result signals OUT is output in the above example, it is possible to calculate and output the moving average of three or more measurement result signals OUT (e.g., measurement result signal OUT acquired in the current measurement and measurement result signals OUT acquired in a plurality of immediately preceding measurements).

### Second Embodiment

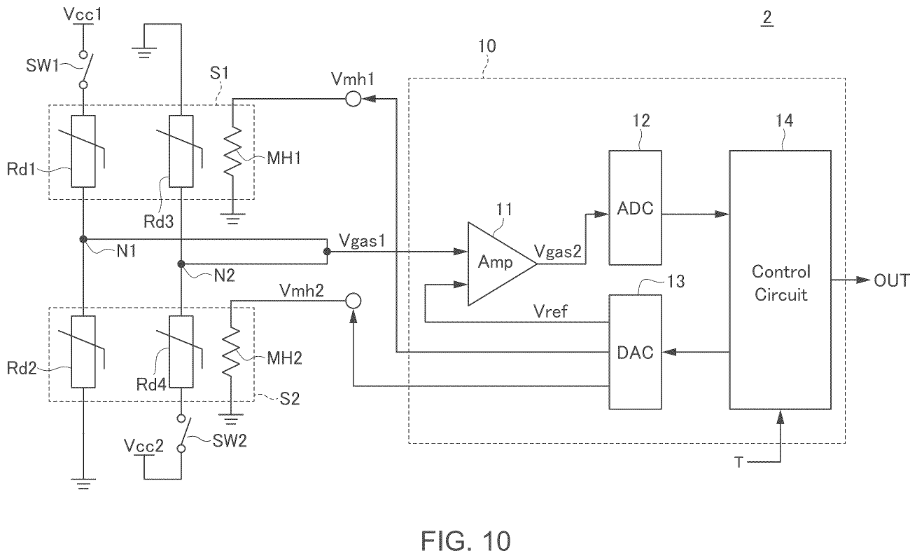

FIG. 10 is a circuit diagram illustrating the configuration of a gas sensor 2 according to a second embodiment of the technology according to the present disclosure.

As illustrated in FIG. 10, the gas sensor 2 according to the second embodiment differs from the gas sensor 1 according to the first embodiment in that the switches SW3 and SW4 are omitted with the result that the other ends of the thermistors Rd2 and Rd3 are directly connected to the ground lines. Other basic configurations are the same as those of the gas sensor 1 according to the first embodiment, so the same reference numerals are given to the same elements, and overlapping description will be omitted.

The gas sensor 2 according to the second embodiment performs the same operation content as that of the gas sensor 1 according to the first embodiment except that it does not control the switches SW3 and SW4. That is, the switches SW3 and SW4 are absent in the gas sensor 2 according to the second embodiment, so that the node N2 is connected to the ground line through the thermistor Rd3 for reference in the period T1, and the node N1 is connected to the ground line through the thermistor Rd2 for reference in the period T2. However, the thermistor Rd3 is heated to about 150° C. in the period T1, and the thermistor Rd2 is heated to about 150° C. in the period T2, while the resistance values of the thermistors Rd2 and Rd3 for reference are designed so as to fall within a predetermined resistance range when the thermistors Rd2 and Rd3 are heated to about 300° C. Therefore, the resistance values of the thermistors Rd2 and Rd3 heated to about 150° C. have a negative temperature coefficient of resistance and thus are sufficiently high, so that the influence of the thermistor Rd3 in the period T1 and the influence of the thermistor Rd2 in the period T2 become negligeable.

As described above, owing to the omission of the switches SW3 and SW4, circuit scale can be further simplified in the gas sensor 2 according to the second embodiment.

### Third Embodiment

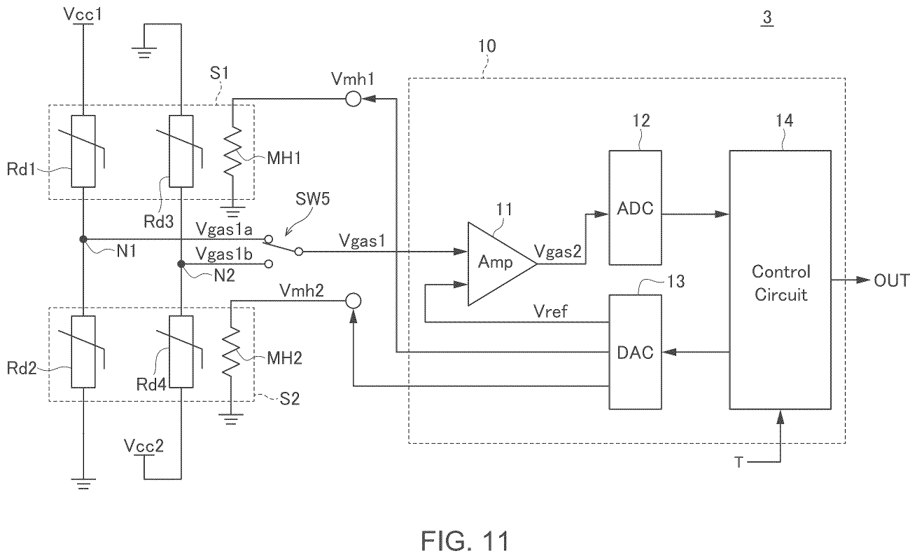

FIG. 11 is a circuit diagram illustrating the configuration of a gas sensor 3 according to a third embodiment of the technology according to the present disclosure.

As illustrated in FIG. 11, in the gas sensor according to the third embodiment, the switches SW1 to SW4 are omitted. Accordingly, the other end of the thermistor Rd1 is directly connected to the line to which the power supply voltage Vcc1 is supplied, the other ends of the thermistors Rd2 and Rd3 are directly connected to their corresponding ground lines, and the other end of the thermistor Rd4 is directly connected to the line to which the power supply voltage Vcc2 is supplied. Further, in the gas sensor 3 according to the third embodiment, a switch SW5 connecting one of the nodes N1 and N2 to the amplifier 11 is additionally provided. The switch SW5 is controlled by the signal processing circuit 10. When the node N1 is selected, a detection voltage Vgas1a appearing at the node N1 is supplied to the amplifier 11 as the detection voltage Vgas1, while when the node N2 is selected, a detection voltage Vgas1b appearing at the node N2 is supplied to the amplifier 11 as the detection voltage Vgas1. The signal processing circuit 10 controls the switch SW5 to select the node N1 in the period T1 and controls the switch SW5 to select the node N2 in the period T2. Other basic configurations are the same as those of the gas sensor 1 according to the first embodiment, so the same reference numerals are given to the same elements, and overlapping description will be omitted.

As described above, in the gas sensor 3 according to the third embodiment, the nodes N1 and N2 are exclusively connected, through the switch SW5, to the amplifier 11 without being short-circuited, thus preventing the detection voltage Vgas1a appearing at the node N1 and the detection voltage Vgas1b appearing at the node N2 from influencing each other.

### Fourth Embodiment

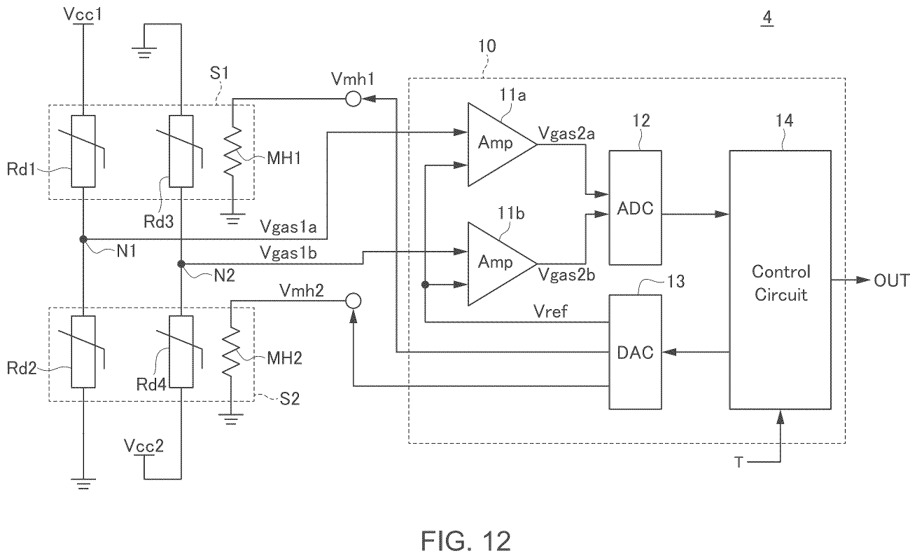

FIG. 12 is a circuit diagram illustrating the configuration of a gas sensor 4 according to a fourth embodiment of the technology according to the present disclosure.

As illustrated in FIG. 12, in the gas sensor 4 according to the fourth embodiment, the detection voltage Vgas1a appearing at the node N1 is supplied to an amplifier 11a, and the detection voltage Vgas1b appearing at the node N2 is supplied to an amplifier 11b. The amplifier 11a is a differential amplifier, for example, and compares the detection voltage Vgas1a and the reference voltage Vref, for example, to generate an amplified detection voltage Vgas2a. The amplifier 11b is a differential amplifier, for example, and compares the detection voltage Vgas1b and the reference voltage Vref, for example, to generate an amplified detection voltage Vgas2b. The detection voltages Vgas2a and Vgas2b are input to the AD converter 12. The AD converter 12 A-D converts the input detection voltages Vgas2a and Vgas2b into digital detection voltages Vgas2a and Vgas2b and supplies the digital detection voltages Vgas2a and Vgas2b to the control circuit 14. Other basic configurations are the same as those of the gas sensor 3 according to the third embodiment, so the same reference numerals are given to the same elements, and overlapping description will be omitted.

As described above, in the gas sensor 4 according to the fourth embodiment, the nodes N1 and N2 are connected to the different amplifiers 11a and 11b, respectively, without being short-circuited, thus preventing the detection voltage Vgas1a appearing at the node N1 and the detection voltage Vgas1b appearing at the node N2 from influencing each other.

### Fifth Embodiment

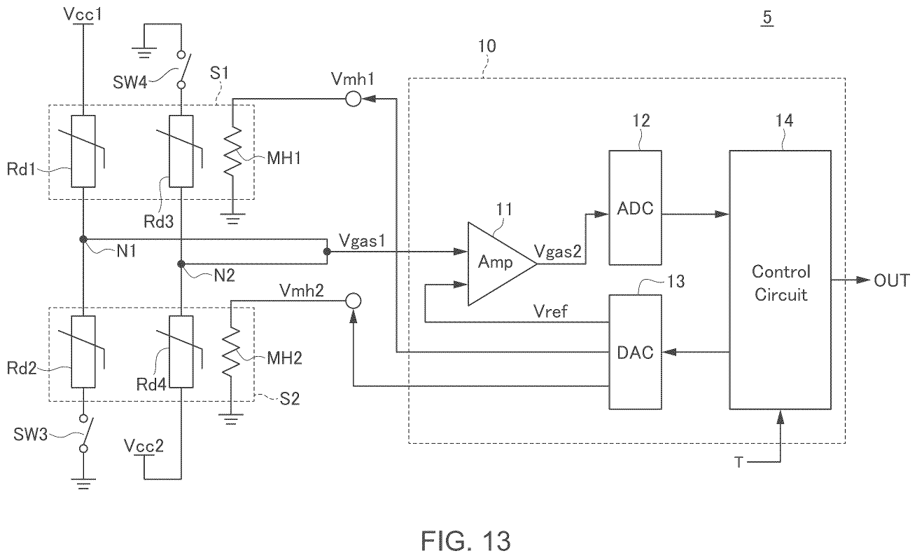

FIG. 13 is a circuit diagram illustrating the configuration of a gas sensor 5 according to a fifth embodiment of the technology according to the present disclosure.

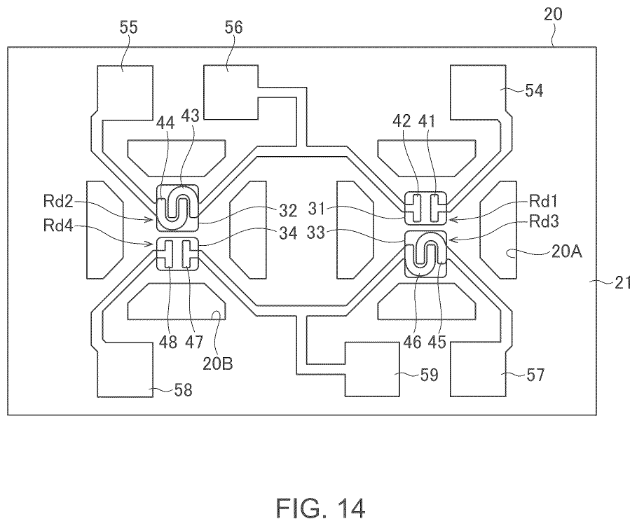

As illustrated in FIG. 13, the gas sensor 5 according to the fifth embodiment differs from the gas sensor 1 according to the first embodiment in that the switches SW1 and SW2 are omitted with the result that the other ends of the thermistors Rd1 and Rd4 are directly connected to the lines to which the power supply voltages Vcc1 and Vcc2 are supplied, respectively, and that the thermistor films 31 to 34 are each made of a material having a positive temperature coefficient of resistance. Further, as illustrated in FIG. 14, in the gas sensor 5 according to the fifth embodiment, the counter electrodes 43 and 44 (also counter electrodes 45 and 46) are so shaped as to be engaged with each other in a comb-like shape, while the counter electrodes 41 and 42 (also counter electrodes 47 and 48) are so shaped as to linearly face each other. As a result, the opposing length between the counter electrodes 43 and 44 (also counter electrodes 45 and 46) is longer than the opposing length between the counter electrodes 41 and 42 (also counter electrodes and 47 and 48). Further, the inter-electrode distance (opposing width) between the counter electrodes 43 and 44 (also counter electrodes 45 and 46) is smaller than the inter-electrode distance (opposing width) between the counter electrodes 41 and 42 (also counter electrodes 47 and 48). Thus, when the thermistor films 31 to 34 having substantially the same resistivity are heated to substantially the same temperature, the resistance value of the thermistor film 31 positioned between the counter electrodes 41 and 42 and that of the thermistor film 34 positioned between the counter electrodes 47 and 48 become higher than the resistance value of the thermistor film 32 positioned between the counter electrodes 43 and 44 and that of the thermistor film 33 positioned between the counter electrodes 45 and 46. Other basic configurations are the same as those of the gas sensor 1 according to the first embodiment, so the same reference numerals are given to the same elements, and overlapping description will be omitted.

The gas sensor 5 according to the fifth embodiment performs the same operation content as that of the gas sensor 1 according to the first embodiment except that it does not control the switches SW1 and SW2. That is, the switches SW1 and SW2 are absent in the gas sensor 5 according to the fifth embodiment, so that the node N2 is connected, through the thermistor Rd4 for detection, to the line to which the power voltage Vcc2 is supplied in the period T1, and the node N1 is connected, through the thermistor Rd1 for detection, to the line to which the power voltage Vcc1 is supplied in the period T2. However, the thermistor Rd4 is heated to about 300° C. in the period T1, and the thermistor Rd1 is heated to about 300° C. in the period T2, while the resistance values of the thermistors Rd1 and Rd4 for detection are designed so as to fall within a predetermined resistance range when the thermistors Rd1 and Rd4 are heated to about 150° C. Therefore, the resistance values of the thermistors Rd1 and Rd4 heated to about 300° C. have a positive temperature coefficient of resistance and thus are sufficiently high, so that the influence of the thermistor Rd4 in the period T1 and the influence of the thermistor Rd1 in the period T2 become negligeable.

As exemplified in the gas sensor 5 according to the fifth embodiment, the thermistor films 31 to 34 may each be made of a material having a positive temperature coefficient of resistance.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, although a thermistor is used as the temperature-sensitive element, the present invention is not limited to this.

The technology according to the present disclosure includes the following configuration examples, but not limited thereto.

A gas sensor according to an aspect of the present disclosure includes: a first series circuit including a first temperature-sensitive element and a second temperature-sensitive element connected in series; a second series circuit including a third temperature-sensitive element and a fourth temperature-sensitive element connected in series; a first power supply circuit configured to apply voltage to the first series circuit; a second power supply circuit configured to apply voltage to the second series circuit; and a signal processing circuit. The signal processing circuit is configured to: detect a first detection voltage appearing at a first node at which the first temperature-sensitive element and the second temperature-sensitive element are connected in a first period during which the first temperature-sensitive element and a third temperature-sensitive element are heated to a first temperature range and the second temperature-sensitive element and the fourth temperature-sensitive element are heated to a second temperature range; detect a second detection voltage appearing at a second node at which the third temperature-sensitive element and the fourth temperature-sensitive element are connected in a second period during which the first temperature-sensitive element and the third temperature-sensitive element are heated to the second temperature range and the second temperature-sensitive e element and the fourth temperature-sensitive element are heated to the first temperature range; and calculate a concentration of a gas to be detected based on the first detection voltage and the second detection voltage. This can make thermal histories of the first and second temperature-sensitive elements coincide with each other, make thermal histories of the third and fourth temperature-sensitive elements coincide with each other, and can achieve measurement of the gas to be detected in both the first and second periods.

In the above gas sensor, the resistance values of each of the first temperature-sensitive element and the fourth temperature-sensitive element falls within a first resistance range when the first temperature-sensitive element and the fourth temperature-sensitive element are heated to the first temperature range, and the resistance value of each of the second temperature-sensitive element and the third temperature-sensitive element falls within a second resistance range when the second temperature-sensitive element and the third temperature-sensitive element are heated to the second temperature range. This makes it possible to detect the concentration of a non-flammable gas (e.g., CO₂ gas) contained in the atmosphere.

In this case, the first resistance range and the second resistance range may overlap each other. This can achieve a wide dynamic range.

The above gas sensor may further include a first heater configured to heat the first temperature-sensitive element and the third temperature-sensitive element in common, and a second heater configured to heat the second temperature-sensitive element and the fourth temperature-sensitive element in common. This makes it possible to heat the first and third temperature-sensitive elements to the same temperature and to heat the second and fourth temperature-sensitive elements to the same temperature.

In the above gas sensor, the first temperature-sensitive element may include a first portion of a first thermistor film heated by the first heater and a pair of first counter electrodes facing each other through the first portion of the first thermistor film, the second temperature-sensitive element may include a first portion of a second thermistor film heated by the second heater and a pair of second counter electrodes facing each other through the first portion of the second thermistor film, the third temperature-sensitive element may include a first portion of a third thermistor film heated by the first heater and a pair of third counter electrodes facing each other through the first portion of the third thermistor film, and the fourth temperature-sensitive element may include a first portion of a fourth thermistor film heated by the second heater and a pair of fourth counter electrodes facing each other through the first portion of the fourth thermistor film. This can enhance insulation between the first counter electrodes and the third counter electrodes and between the second counter electrodes and the fourth counter electrodes.

In the above gas sensor, the first temperature-sensitive element may include a first portion of a first thermistor film heated by the first heater and a pair of first counter electrodes facing each other through the first portion of the first thermistor film, the second temperature-sensitive element may include a first portion of a second thermistor film heated by the second heater and a pair of second counter electrodes facing each other through the first portion of the second thermistor film, the third temperature-sensitive element may include a second portion of the first thermistor film and a pair of third counter electrodes facing each other through the second portion of the first thermistor film, and the fourth temperature-sensitive element may include a second portion of the second thermistor film and a pair of fourth counter electrodes facing each other through the second portion of the second thermistor film. This can miniaturize the first to fourth temperature-sensitive elements.

In the above gas sensor, each of the first to fourth temperature-sensitive elements may have a negative temperature coefficient of resistance, the second temperature range may be higher than the first temperature range, the opposing length between the first counter electrodes may be larger than the opposing length between the second counter electrodes, and the opposing length between the fourth counter electrodes may be larger than the opposing length between the third counter electrodes. This can make the resistance value of the first temperature-sensitive element lower than the resistance value of the second temperature-sensitive element and make the resistance value of the fourth temperature-sensitive element lower than the resistance value of the third temperature-sensitive element.

In the above gas sensor, each of the first to fourth temperature-sensitive elements may have a negative temperature coefficient of resistance, the second temperature range may be higher than the first temperature range, the inter-electrode distance between the first counter electrodes may be smaller than the inter-electrode distance between the second counter electrodes, and the inter-electrode distance between the fourth counter electrodes may be smaller than the inter-electrode distance between the third counter electrodes. This can make the resistance value of the first temperature-sensitive element lower than the resistance value of the second temperature-sensitive element and make the resistance value of the fourth temperature-sensitive element lower than the resistance value of the third temperature-sensitive element.

In the above gas sensor, each of the first to fourth temperature-sensitive elements may have a positive temperature coefficient of resistance, the second temperature range may be higher than the first temperature range, the opposing length between the second counter electrodes may be larger than the opposing length between the first counter electrodes, and the opposing length between the third counter electrodes may be larger than the opposing length between the fourth counter electrodes. This can make the resistance value of the second temperature-sensitive element lower than the resistance value of the first temperature-sensitive element and make the resistance value of the third temperature-sensitive element lower than the resistance value of the fourth temperature-sensitive element.

In the above gas sensor, each of the first to fourth temperature-sensitive elements may have a positive temperature coefficient of resistance, the second temperature range may be higher than the first temperature range, the inter-electrode distance between the second counter electrodes may be smaller than the inter-electrode distance between the first counter electrodes, and the inter-electrode distance between the third counter electrodes may be smaller than the inter-electrode distance between the fourth counter electrodes. This can make the resistance value of the second temperature-sensitive element lower than the resistance value of the first temperature-sensitive element and make the resistance value of the third temperature-sensitive element lower than the resistance value of the fourth temperature-sensitive element.

In the above gas sensor, each of the first to fourth temperature-sensitive elements may have a negative temperature coefficient of resistance, the second temperature range may be higher than the first temperature range, the first power supply circuit may include a first switch connected between a first power supply line to which a first power supply voltage is supplied or a ground line and the first temperature-sensitive element, and the second power supply circuit may include a second switch connected between a second power supply line to which a second power supply voltage is supplied or a ground line and the fourth temperature-sensitive element. This allows the first temperature-sensitive element to be separated from the first power supply line or ground line and allows the fourth temperature-sensitive element to be separated from the second power supply line or ground line. In this case, the first node and the second node may be short-circuited, and the signal processing circuit may turn on the first switch and turn off the second switch in the first period and may turn off the first switch and turn on the second switch in the second period. This allows the fourth temperature-sensitive element to be separated from the second power supply line or ground line in the first period and allows the first temperature-sensitive element to be separated from the first power supply line or ground line in the second period.

In the above gas sensor, each of the first to fourth temperature-sensitive elements may have a positive temperature coefficient of resistance, the second temperature range may be higher than the first temperature range, the first power supply circuit may include a first switch connected between a first power supply line to which a first power supply voltage is supplied or a ground line and the second temperature-sensitive element, and the second power supply circuit may include a second switch connected between a second power supply line to which a second power supply voltage is supplied or a ground line and the third temperature-sensitive element. This allows the second temperature-sensitive element to be separated from the first power supply line or ground line and allows the third temperature-sensitive element to be separated from the second power supply line or ground line. In this case, the first node and the second node may be short-circuited, and the signal processing circuit may turn on the first switch and turn off the second switch in the first period and may turn off the first switch and turn on the second switch in the second period. This allows the third temperature-sensitive element to be separated from the second power supply line or ground line in the first period and allows the second temperature-sensitive element to be separated from the first power supply line or ground line in the second period.

In the above gas sensor, the first power supply circuit may include a first switch connected between one of a first power supply line to which a first power supply voltage is supplied and a ground line and the first temperature-sensitive element and a third switch connected between the other one of the first power supply line and the ground line and the second temperature-sensitive element, and the second power supply circuit may include a second switch connected between one of a second power supply line to which a second power supply voltage is supplied and the ground line and the fourth temperature-sensitive element and a fourth switch connected between the other one of the second power supply line and ground line and the third temperature-sensitive element. This allows the first temperature-sensitive element to be separated from one of the first power supply line and ground line, allows the second temperature-sensitive element to be separated from the other one of the first power supply line and ground line, allows the third temperature-sensitive element to be separated from one of the second power supply line and ground line, and allows the fourth temperature-sensitive element to be separated from the other one of the second power supply line and ground line. In this case, the first node and the second node may be short-circuited and the signal processing circuit may be configured to turn on the first switch and the third switch and turn off the second switch and the fourth switch in the first period and turn off the first switch and the third switch and turn on the second switch and the fourth switch in the second period. Thus, the second node is opened in the first period, and the first node is opened in the second period.

In the above gas sensor, the signal processing circuit may be configured to calculate the moving average of the concentration of the gas to be detected acquired based on the first detection voltage and the concentration of the gas to be detected acquired based on the second detection voltage. Thus, even when there occurs a certain offset in the first and second detection voltages, the offset can be cancelled.

What is claimed is:

1. A gas sensor comprising:
a first series circuit including a first temperature-sensitive element and a second temperature-sensitive element connected in series;
a second series circuit including a third temperature-sensitive element and a fourth temperature-sensitive element connected in series;
a first power supply circuit configured to apply voltage to the first series circuit;
a second power supply circuit configured to apply voltage to the second series circuit; and
a signal processing circuit configured to:
detect a first detection voltage appearing at a first node at which the first temperature-sensitive element and the second temperature-sensitive element are connected in a first period during which the first temperature-sensitive element and a third temperature-sensitive element are heated to a first temperature range and the second temperature-sensitive element and the fourth temperature-sensitive element are heated to a second temperature range;
detect a second detection voltage appearing at a second node at which the third temperature-sensitive element and the fourth temperature-sensitive element are connected in a second period during which the first temperature-sensitive element and the third temperature-sensitive element are heated to the second temperature range and the second temperature-sensitive element and the fourth temperature-sensitive element are heated to the first temperature range; and
calculate a concentration of a gas to be detected based on the first detection voltage and the second detection voltage.

2. The gas sensor as claimed in claim 1,
wherein a resistance value of each of the first temperature-sensitive element and the fourth temperature-sensitive element falls within a first resistance range when the first temperature-sensitive element and the fourth temperature-sensitive element are heated to the first temperature range, and
wherein a resistance value of each of the second temperature-sensitive element and the third temperature-sensitive element falls within a second resistance range when the second temperature-sensitive element and the third temperature-sensitive element are heated to the second temperature range.

3. The gas sensor as claimed in claim 2, wherein the first resistance range and the second resistance range overlap each other.

4. The gas sensor as claimed in claim 1, further comprising:
a first heater configured to heat the first temperature-sensitive element and the third temperature-sensitive element in common; and
a second heater configured to heat the second temperature-sensitive element and the fourth temperature-sensitive element in common.

5. The gas sensor as claimed in claim 4,
wherein the first temperature-sensitive element includes a first portion of a first thermistor film heated by the first heater and a pair of first counter electrodes facing each other through the first portion of the first thermistor film,
wherein the second temperature-sensitive element includes a first portion of a second thermistor film heated by the second heater and a pair of second counter electrodes facing each other through the first portion of the second thermistor film,
wherein the third temperature-sensitive element includes a first portion of a third thermistor film heated by the first heater and a pair of third counter electrodes facing each other through the first portion of the third thermistor film, and
wherein the fourth temperature-sensitive element includes a first portion of a fourth thermistor film heated by the second heater and a pair of fourth counter electrodes facing each other through the first portion of the fourth thermistor film.

6. The gas sensor as claimed in claim 4,
wherein the first temperature-sensitive element includes a first portion of a first thermistor film heated by the first heater and a pair of first counter electrodes facing each other through the first portion of the first thermistor film,
wherein the second temperature-sensitive element includes a first portion of a second thermistor film heated by the second heater and a pair of second counter electrodes facing each other through the first portion of the second thermistor film,
wherein the third temperature-sensitive element includes a second portion of the first thermistor film and a pair of third counter electrodes facing each other through the second portion of the first thermistor film, and
wherein the fourth temperature-sensitive element includes a second portion of the second thermistor film and a pair of fourth counter electrodes facing each other through the second portion of the second thermistor film.

7. The gas sensor as claimed in claim 5,
wherein each of the first to fourth temperature-sensitive elements has a negative temperature coefficient of resistance,
wherein the second temperature range is higher than the first temperature range,
wherein an opposing length between the first counter electrodes is larger than an opposing length between the second counter electrodes, and
wherein an opposing length between the fourth counter electrodes is larger than an opposing length between the third counter electrodes.

8. The gas sensor as claimed in claim 6,
wherein each of the first to fourth temperature-sensitive elements has a negative temperature coefficient of resistance,
wherein the second temperature range is higher than the first temperature range,
wherein an opposing length between the first counter electrodes is larger than an opposing length between the second counter electrodes, and
wherein an opposing length between the fourth counter electrodes is larger than an opposing length between the third counter electrodes.

9. The gas sensor as claimed in claim 5,
wherein each of the first to fourth temperature-sensitive elements has a negative temperature coefficient of resistance,
wherein the second temperature range is higher than the first temperature range,
wherein an inter-electrode distance between the first counter electrodes is smaller inter-electrode distance between the second counter electrodes, and
wherein an inter-electrode distance between the fourth counter electrodes is smaller than an inter-electrode distance between the third counter electrodes.

10. The gas sensor as claimed in claim 6,
wherein each of the first to fourth temperature-sensitive elements has a negative temperature coefficient of resistance,
wherein the second temperature range is higher than the first temperature range,
wherein an inter-electrode distance between the first counter electrodes is smaller than an inter-electrode distance between the second counter electrodes, and
wherein an inter-electrode distance between the fourth counter electrodes is smaller than an inter-electrode distance between the third counter electrodes.

11. The gas sensor as claimed in claim 5,
wherein each of the first to fourth temperature-sensitive elements has a positive temperature coefficient of resistance,
wherein the second temperature range is higher than the first temperature range,
wherein an opposing length between the second counter electrodes is larger than an opposing length between the first counter electrodes, and
wherein an opposing length between the third counter electrodes is larger than an opposing length between the fourth counter electrodes.

12. The gas sensor as claimed in claim 6,
wherein each of the first to fourth temperature-sensitive elements has a positive temperature coefficient of resistance,
wherein the second temperature range is higher than the first temperature range,
wherein an opposing length between the second counter electrodes is larger than an opposing length between the first counter electrodes, and
wherein an opposing length between the third counter electrodes is larger than an opposing length between the fourth counter electrodes.

13. The gas sensor as claimed in claim 5,
wherein each of the first to fourth temperature-sensitive elements has a positive temperature coefficient of resistance,
wherein the second temperature range is higher than the first temperature range,
wherein an inter-electrode distance between the second counter electrodes is smaller than an inter-electrode distance between the first counter electrodes, and
wherein an inter-electrode distance between the third counter electrodes is smaller than an inter-electrode distance between the fourth counter electrodes.

14. The gas sensor as claimed in claim 6,
wherein each of the first to fourth temperature-sensitive elements has a positive temperature coefficient of resistance,
wherein the second temperature range is higher than the first temperature range,
wherein an inter-electrode distance between the second counter electrodes is smaller than an inter-electrode distance between the first counter electrodes, and
wherein an inter-electrode distance between the third counter electrodes is smaller than an inter-electrode distance between the fourth counter electrodes.

15. The gas sensor as claimed in claim 1,
wherein each of the first to fourth temperature-sensitive elements has a negative temperature coefficient of resistance,
wherein the second temperature range is higher than the first temperature range,
wherein the first power supply circuit includes a first switch connected between a first power supply line to which a first power supply voltage is supplied or a ground line and the first temperature-sensitive element, and
wherein the second power supply circuit includes a second switch connected between a second power supply line to which a second power supply voltage is supplied or the ground line and the fourth temperature-sensitive element.

16. The gas sensor as claimed in claim 1,
wherein each of the first to fourth temperature-sensitive elements has a positive temperature coefficient of resistance,
wherein the second temperature range is higher than the first temperature range,
wherein the first power supply circuit includes a first switch connected between a first power supply line to which a first power supply voltage is supplied or a ground line and the second temperature-sensitive element, and
wherein the second power supply circuit includes a second switch connected between a second power supply line to which a second power supply voltage is supplied or the ground line and the third temperature-sensitive element.

17. The gas sensor as claimed in claim 15,
wherein the first node and the second node are short-circuited, and
wherein the signal processing circuit is configured to turn on the first switch and turn off the second switch in the first period and turn off the first switch and turn on the second switch in the second period.

18. The gas sensor as claimed in claim 1,
wherein the first power supply circuit includes:
a first switch connected between one of a first power supply line to which a first power supply voltage is supplied and a ground line and the first temperature-sensitive element; and
a third switch connected between other one of the first power supply line and the ground line and the second temperature-sensitive element, and
wherein the second power supply circuit includes:
a second switch connected between one of a second power supply line to which a second power supply voltage is supplied and the ground line and the fourth temperature-sensitive element; and
a fourth switch connected between other one of the second power supply line and the ground line and the third temperature-sensitive element.

19. The gas sensor as claimed in claim 18,
wherein the first node and the second node are short-circuited, and
wherein the signal processing circuit is configured to turn on the first switch and the third switch and turn off the second switch and the fourth switch in the first period and turn off the first switch and the third switch and turn on the second switch and the fourth switch in the second period.

20. The gas sensor as claimed in claim 1, wherein the signal processing circuit is configured to calculate a moving average of the concentration of the gas to be detected acquired based on the first detection voltage and the concentration of the gas to be detected acquired based on the second detection voltage.
